(19) United States
(12) Patent Application Publication
(10) Pub. No.: US 2007/0073362 A1
(43) Pub. Date: Mar. 29, 2007

(54) Device for administering a treatment in a body cavity of a human

(76) Inventor: Noel Rodney Campbell, Melbourne (AU)

Correspondence Address: LEYDIG VOIT & MAYER, LTD, 700 THIRTEENTH ST. NW, SUITE 300, WASHINGTON, DC, 20005-3960, US

(21) Appl. No.: 11/233,134

(22) Filed: Sep. 23, 2005

(51) Int. Cl. A61N 5/06

(52) U.S. Cl. 607/88

(57) ABSTRACT

A device for administering a treatment in a body cavity of a human patient has a probe sized for insertion into the cavity. A visible red light source is located in the distal end of the probe for emitting a light having a wavelength within a range of 535 to 785 nanometers in to tissue surrounding the cavity. The proximal end of the probe is connected to a flexible neck having a base. A pair of arms extend laterally from the base for supporting the device on a surface.

### BACKGROUND TO THE INVENTION

1. Field of the Invention

The invention relates to a device for administering a treatment in a body cavity of a human patient, and in particular to a device for administering photo-dynamic therapy within a body cavity of a human.

2. Background Information

Photo-dynamic therapy has been widely used in the treatment of many conditions. Recent research in Russia, Ireland and Germany indicates that photo-dynamic therapy may be useful in the treatment of cancer. Light by itself cannot kill cancer cells, however if a patient is pretreated with an agent called a photo-sensitizer and is administered light of a certain wavelength then cancer cells in the body may be killed.

A current problem with photo-dynamic therapy for treatment of cancer is the effective administering of light to localize cancerous areas within the body.

### SUMMARY OF THE INVENTION

It is an object of the present invention to overcome or substantially ameliorate the above problem and/or more generally to provide a device for administering a treatment in a body cavity of a human.

There is disclosed herein a device for administering a treatment in a body cavity of a human comprising a probe having a proximal end, and a distal end insertable in a body cavity of a human and having an energy transmissive outer surface, a flexible neck connected to the proximal end of the probe and having a base, a pair of arms extending from the base of the neck for supporting the device on a surface, and an energy source located in the distal end of the probe for emitting radiant energy from the energy transmissive surface.

Preferably, the energy source is a visible red light source located in the distal end of the probe for emitting visible red light.

Preferably, the visible red light source comprises a plurality of high intensity light emitting diodes (LEDs).

Preferably, the visible red light source emits a light having a wavelength within a range of 535 to 785 nanometers.

Preferably, the visible red light source emits light at a wavelength of 662 nanometers.

Preferably, the distal end of the probe comprises a light transmissive sheath.

Preferably, the neck comprises a plurality of relatively movable articulated elements arranged in sequence along a longitudinal direction of the neck.

Preferably, the probe is sized for insertion into the anus of a human and has a length such that when the proximal end of the probe is outside the body the light transmissive surface is adjacent a location of a prostate gland within the body.

Preferably, the device is used for photo-dynamic treatment of cancer using visible red light according to one of the following protocols:
i) a one hour treatment every day, or
ii) a one hour treatment three times a week on alternative days per week.
   Further aspects of the invention will become apparent from the following description.

### BRIEF DESCRIPTION OF THE DRAWINGS

A preferred form of the present invention will now be described by way of example with reference to the accompanying drawings, wherein:

FIG. 1 is a perspective view of a device for administering a treatment in a body cavity of a human,

FIG. 2 is a side view of the device, and

FIG. 3 is a top view device.

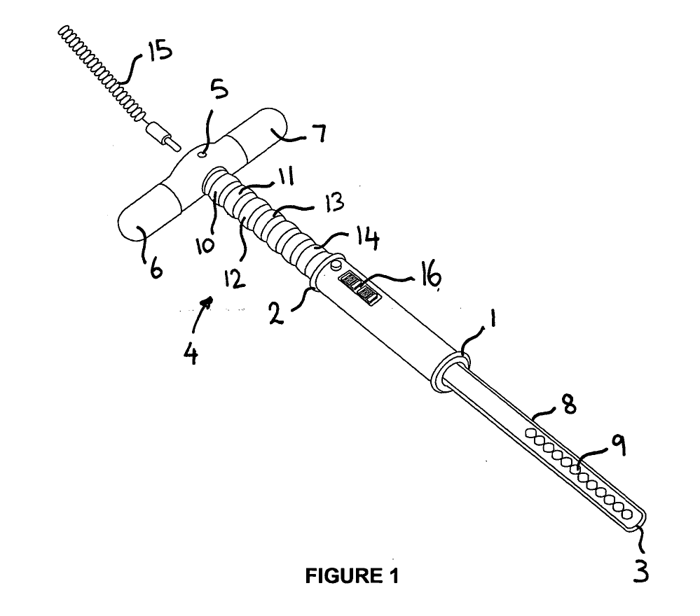

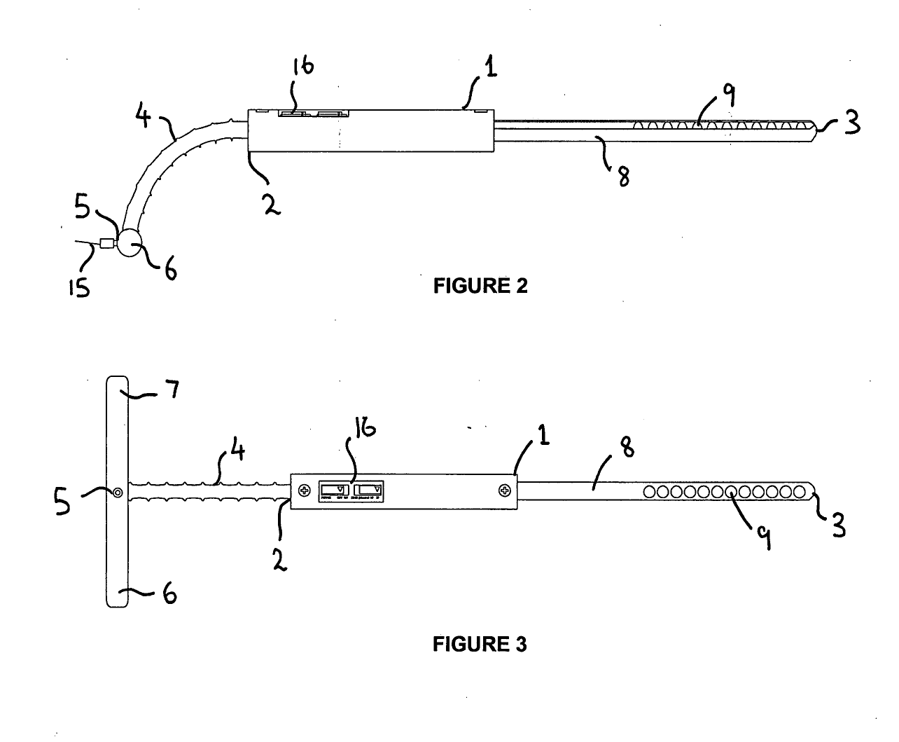

### DESCRIPTION OF THE PERFERRED EXAMPLE

In the drawings there is depicted a device for administering a light treatment in the anus of a human patient. The device comprises a probe 1 having a proximal end 2, and a distal end 3 sized for insertion into the anus of patient. A flexible neck 4 is connected to the proximal end 2 of the probe 1. A base 5 at the opposite end of the flexible neck 4 has two laterally extending arms 6, 7 for supporting the device on a flat surface. The outer surface of the distal end 3 of the probe has a light transmissive sheath in the form of a glass tube 8 that allows easy insertion into the body cavity. A visible red light source 9 is located in the distal end 3 of the probe 1 for emitting visible red light through the glass tube 8.

Typically, the prostate gland is located adjacent the bowel wall up the anal passage. In the preferred embodiment the probe is approximately 16 mm in diameter and 150 in length.

The length of the probe 1 is such that when the probe 1 is inserted in the anus with the proximal end 2 outside the body the light transmissive sheath 8 is adjacent a location of the prostate gland within the anal passage. Visible red light emitted from the light source 9 is delivered to the prostate gland and to tissue around the prostate gland.

The flexible neck 4 permits the patient to lie on a bed with the probe 1 inserted in the anus and the device supported on the bed surface by the base 5 and arms 6, 7. The flexible neck 4 consists of a plurality of relatively movable articulated elements 10, 11, 12, 13 . . . 14 arranged in sequence along the longitudinal direction of the neck 4.

The lateral arms 6, 7 can be taped to the bed surface or positioned under the patient's thighs to prevent movement of the probe 1 during treatment.

The visible red light source comprises a row of twelve high intensity light emitting diodes (LEDs) mounted on a support member positioned within the probe 1. In the preferred embodiment the LEDs are model L660-10U High Bright Red LED Lamps from Epitex (www.epitex.com) of Japan that emit light at a peak wavelength of 660 nanometers. However, treatment can be effected with a light source that emits a light having a wavelength within a range of 535 to 785 nanometers.

The light source is connected an electrical inlet in the base 5 into which an electrical supply 15 is connected for supplying energy for operation of the light source 9. On/off and control switches 16 are provided for controlling operation of the device. The switches 16 operate a timer which allows for continuous treatment, 30 minutes treatment or 60 minutes treatment.

The device can be used for a variety of photo-dynamic treatments within a body cavity of a human. In particular it is useful for photo-dynamic treatment of prostate cancer in males according to one of the following treatment protocols:
a one hour treatment every day, or
a one hour treatment three times a week on alternative days per week,
   continued until there have been three continuous monthly tests demonstrating a return to normal of tumour markers.

It should be appreciated that modifications and alternations obvious to those skilled in the art are not to be considered as beyond the scope of the present invention. For example, in the preferred embodiment the distal end 3 of the probe has a light transmissive sheath 8. In an alternative embodiment the light transmissive region may be a narrow window for specific directional emission of light.

The flexible neck 4 in the preferred embodiment comprises articulated elements. In an alternative embodiment the flexible neck can be a single sheath of flexible material.

The preferred embodiment has LEDs for emitting visible red light at a wavelength of 662 nanometers for use in treating prostate cancer. In alternative embodiments the probe may include other wavelengths or other energy source for alternative treatments such as infra-red or ultrasonic energy sources. The probe may also be sized for insertion into other body cavities such as the mouth, nose, ear or vagina for treatment of other conditions and cancer types including, but not limited to, rectal cancer, cervical cancer, cancer of the tongue, cancer of the nasal passages & sinuses. The device can also be used for preventative treatments for these conditions.

The illustrated embodiment uses a single row of twelve LEDs. In alternative embodiments there may be more or less LEDs. For examples one embodiment may have twenty-four LEDs in an single row or two rows of twelve.

What is claimed is:

1. A device for administering a treatment in a body cavity of a human comprising:
a probe having a proximal end, and a distal end insertable in a body cavity of a human and having an energy transmissive outer surface,
a flexible neck connected to the proximal end of the probe and having a base,
a pair of arms extending from the base of the neck for supporting the device on a surface, and
an energy source located in the distal end of the probe for emitting radiant energy from the energy transmissive surface.

2. The device of claim 1 wherein the energy source is a visible red light source located in the distal end of the probe for emitting visible red light.

3. The device of claim 2 wherein the visible red light source comprises a plurality of high intensity light emitting diodes (LEDs).

4. The device of claim 2 wherein the visible red light source emits a light having a wavelength within a range of 535 to 785 nanometers.

5. The device of claim 2 wherein the visible red light source emits light at a wavelength of 662 nanometers.

6. The device of claim 1 wherein the distal end of the probe comprises a light transmissive sheath.

7. The device of claim 1 wherein the neck comprises a plurality of relatively movable articulated elements arranged in sequence along a longitudinal direction of the neck.

8. The device of claim 1 wherein the probe is sized for insertion into the anus of a human and has a length such that when the proximal end of the probe is outside the body the light transmissive surface is adjacent a location of a prostate gland within the body.

9. A device for administering a treatment to a prostate gland of a human comprising:
a probe having a proximal end, a distal end having a light transmissive outer surface and sized for insertion into the anus of a human and having a length such that when the proximal end of the probe is outside the body the light transmissive surface is adjacent a location of a prostate gland within the body
a flexible neck connected to the proximal end of the probe and having a base,
a pair of arms extending from the base of the neck for supporting the device on a surface, and
a visible red light source located in the distal end of the probe for emitting a light having a wavelength within a range of 535 to 785 nanometers in to tissue around the prostate gland.

10. A device insertable into a body cavity of a human patient for photo-dynamic treatment of cancer using visible red light according to one of the following protocols:
i) a one hour treatment every day, or
ii) a one hour treatment three times a week on alternative days per week.

11. The device of claim 11 when used to treat prostate cancer in male patients.
